(19) United States
(12) Patent Application Publication
(10) Pub. No.: US 2004/0001597 A1
(43) Pub. Date: Jan. 1, 2004

(54) Audio communication system and method with improved acoustic characteristics

(75) Inventor: Trygve F. Marton, Oslo (NO)

Correspondence Address: OBLON SPIVAK MCCLELLAND MAIER & NEUSTADT, 1755 JEFFERSON DAVIS HIGHWAY, FOURTH FLOOR, ARLINGTON, VA, 22202, US

(73) Assignee: TANDBERG ASA, Lysaker (NO)

(21) Appl. No.: 10/184,986

(22) Filed: Jul. 1, 2002

(51) Int. Cl. A61F 11/06; G10K 11/16

(52) U.S. Cl. 381/71.1; 379/406.01

(57) ABSTRACT

A communication system for transferring audio signals includes an audio presenting unit configured to produce a sound wave, an audio capturing unit configured to capture a sound wave, and an acoustic echo canceller unit connected to the audio presenting unit and the audio capturing unit. The audio echo canceller unit includes a model of an acoustic wave, in which the model produces an echo estimate which is subtracted from the captured sound wave which includes an echo, and the audio presenting unit includes a sound producing device connected and controlled by a current controlled source. The communication system further includes a compensator configured to provide an electrical damping for the sound producing device. Alternatively, the sound producing device is connected and controlled by a weighted combination of voltage and current controlled source.

### BACKGROUND OF THE INVENTION

1. Field of the Invention

The present invention relates to an audio communication system and method with improved acoustic characteristics, and particularly to a video conferencing system including an audio subsystem and a video subsystem both distributed at least at two different locations.

2. Discussion of the Background

In a conventional conferencing system setup that uses loudspeakers, two or more communication units are placed at separate sites. A signal transmitted from one site to another site by the conferencing system experiences several delays, among these delays being a transmission delay and a processing delay. For a video conferencing system, the processing delay for video signals is considerably larger than the processing delay for the audio signals. Because the video and audio signals have to be presented simultaneously, in phase, a lipsync delay is purposefully introduced to the audio signal, both in the transmitting and receiving signal paths in order to compensate for the longer video signal delay.

In a conventional conferencing system, an audio capturing system, usually a microphone, captures a sound wave at a site A, and transforms the sound wave into a first audio signal. The first audio signal is transmitted to a site B, where an audio presenting system, usually a television set or an amplifier and loudspeaker, reproduces the original sound wave by converting the first audio signal generated at site A into the sound wave. The produced sound wave at site B, is captured partially by the audio capturing system at site B, converted to a second audio signal, and transmitted back to the system at site A. This problem of having a sound wave captured at one site, transmitted to another site, and then transmitted back to the initial site is referred to an acoustic echo. In its most severe manifestation, the acoustic echo might cause the communication system to howl, when the loop gain exceeds unity. The acoustic echo also causes the participants at site A to hear themselves and causes the participants at site B to hear themselves, making a conversation over the conferencing system difficult, especially if there are delays in the system setup. For the video conferencing system, there are delays due to both processing and transmission delays, and therefore, the acoustic echo is more severe than for the audio conferencing system.

FIG. 1 shows a conventional conferencing system setup. For simplicity, FIG. 1 shows the conferencing system setup distributed at two sites, A and B. The two sites are connected through a transmission channel 1300 and each site has a loudspeaker 1100 and 1200, respectively, and a microphone 1111 and 1211, respectively. The arrows in FIG. 1 indicate the direction of propagation for an acoustic signal, usually from the microphone to the loudspeaker.

One approach to alleviate the acoustic echo is to compensate for the acoustic echo. In a high quality communication system, the compensation of the acoustic echo is normally achieved by an acoustic echo canceller. The acoustic echo canceller is a stand-alone device or an integrated part in the case of the communication system. The acoustic echo canceller models the acoustic signal transmitted from site A to site B, for example, using a linear/nonlinear mathematical model and then substracts the mathematically modulated acoustic signal from the acoustic signal transmitted from site B to site A. In more detail, referring for example to the conferencing system at site B, the acoustic echo canceller passes the first acoustic signal from site A through the mathematical model, calculates an estimate of the echo signal, subtracts the estimated echo signal from the second audio signal captured at site B, and transmits back the second audio signal, less the estimated echo to site A. More sophisticated echo canceller systems may further use an estimation error, i.e., a difference between the estimated echo and the actual echo, to update or adapt the mathematical model to a background noise and changes of the environment, at a position where the sound is captured by the audio capturing device. The mathematical model including the estimation, subtraction, and addition of audio signals is referred to hereinafter as the echo compensator.

However, a problem with the acoustic echo canceller and echo compensator is that their performances are dependent on how well the mathematical model estimates the audio echo (estimate audio echo) to match the actual echo signal of the communication system. Any mismatch between the actual echo and the estimated echo causes a residual echo which deteriorates the quality of the sound that is transmitted from one site to another site. As the communication delay increases, the deterioration produced by the residual echo increases. The conventional approach to further reduce the residual echo is to add voice switching (attenuation) to some extent, creating therefore a system which is neither full nor half duplex. The part of the echo canceller that adds voice switching is hereinafter referred to as the nonlinear processor.

Some models have addressed the problem of compensating the mismatch between the estimated echo and the actual echo and therefore reducing the residual echo by improving the mathematical models used for describing the acoustic signal corresponding to a sound wave. A problem with these models is that they become complicated and costly because the mathematical model becomes nonlinear and therefore involves sophisticated algorithms and refined hardware capabilities. In addition, as the model becomes highly nonlinear, the processing time of the model increases, and therefore, more delays are introduced. For example, in U.S. Pat. No. 5,937,060, a residual echo suppression system is described for hands-free cellular telephones for use in automobiles. The residual echo suppression system replaces a remaining echo signal by reshaping the spectrum of the acoustic signal so that the shape of the spectrum matches the background noise spectrum. In another example, in U.S. Pat. No. 5,737,408, an echo cancelling system suitable for voice conference is described as capable of cancelling echos with accuracy. The solution proposed by this work has an echo cancelling system with two echo cancellers for cancelling a channel echo and a room echo. In a further example, in U.S. Pat. No. 6,198,819, an echo canceller having an improved nonlinear processor is presented. The echo canceller with the improved nonlinear processor describes a nonlinear processor which inhibits a dynamic setting of certain values in double-talk situations or locks a value of an echo return loss measurement after a predetermined number of consecutive echo loss measurements.

However, as recognized by Stenger and Kellerman in “Nonlinear Acoustic Echo Cancellation with Fast Converging Memoryless Preprocessor,” presented at 2000 IEEE International Conference on Acoustics, Speech and Signal Processing, Jun. 5-9, 2000, Istanbul, an improved echo cancellation performance can be achieved by applying a nonlinear acoustic echo model. Thus, a nonlinear acoustic echo cancellation system requires a fast convergence and therefore a complicated and costly system. In addition, extra delays are introduced due to the nonlinearity of the model.

A problem with a linear acoustic echo cancellation system is that a moving voice-coil loudspeaker which is widely used as a transducer for audio frequencies in a conferencing system, is an imperfect device, and generates nonlinear signals and distortion, two effects that greatly deteriorate the quality of the acoustic echo cancellation system.

Hsu and Poornima in “Electronic Damping for Dynamic Drivers in Vented Enclosures,” J. Audio Eng. Soc., Vol. 47, No. 1/2, January/February, have described a high-power loudspeaker operated by a current drive mechanism instead of a voltage drive mechanism having the advantage that the current drive mechanism eliminates the performance dependency of the loudspeaker on the voice-coil resistance and also on the coil inductive effects, which give rise to high-frequency distortion. However, a problem, as recognized by the present inventor, with the loudspeaker having the current drive mechanism is a lack of electrical damping. Hsu and Poornima describe that “the current drive, inherently removes electrical damping caused by a low amplifier source impedance,” at page 32, col. 1, second paragraph. Hsu and Poornima propose a cone velocity feedback for substituting the lack of electrical damping. For achieving the cone velocity feedback, a method is proposed for coupling the loudspeaker of the system with another loudspeaker whose cone and dome have been removed. Thus, instead of having one loudspeaker, the system proposed by Hsu and Poornima has at least two loudspeakers connected by a rigid tube. As recognized by the present inventor, this consequently changes the mass and the sound quality of the system, and increases the volume occupied by the communication system. In addition, a pair of loudspeakers mechanically connected for providing electrical damping is prone to failure, and therefore not reliable.

### SUMMARY OF THE INVENTION

It is an object of the present invention to avoid the above-identified and other limitations of conventional systems and methods. Thus a feature of the present audio communication system and method is to provide improved acoustic characteristics particularly for a videoconferencing system that is both inexpensive and reliable, and which can be based on linear calculation models for echo cancelling and standard loudspeakers.

The present invention is based on a realization that echo cancellation can be improved by designing an acoustic system which better matches the mathematical model presently used in echo cancellers. For a linear mathematical model, the acoustic system should ideally be purely linear, i.e., the nonlinearity of the acoustic system should be as low as possible.

The present invention is further based on a realization that the most nonlinear system component is the loudspeaker. Reducing the source of the nonlinearity of the loudspeaker is proposed as an alternative to reducing the nonlinearity by using a more expensive loudspeaker design.

An object of the present invention is to adapt an interface between the loudspeaker and an amplifier of the communication system in such a way that the loudspeaker is current controlled (high amplifier output impedance) instead of voltage controlled (low amplifier output impedance) or a weighted combination (hybrid) of voltage and current controlled sources to drive the loudspeaker.

Harmonic distortion and variation in sensitivity caused by voice-coil thermal effects are two severe nonlinear characteristics that degrade the performance of the echo compensator and both are present in regular voltage controlled loudspeakers. These effects have only small consequences when the loudspeaker is used for audio presenting only, like in music reproduction systems or televisions and the applied standard for interfacing between amplifiers and loudspeakers is voltage control. By adapting the loudspeaker for current control instead of voltage control, the harmonic distortion is reduced, while the thermal effects are completely eliminated.

However, current control inherently removes the electrical damping caused by a low impedance source impedance; this inconvenience of current control is solved by the present invention by introducing digital compensation of the acoustic signal (which functions as damping) prior to the digital-to-analog converter.

Another object of the present invention is an audio communication system and method for transferring audio signals, including an audio presenting unit configured to produce a first sound wave; an audio capturing unit configured to capture a second sound wave; and an acoustic echo canceller unit connected to the audio presenting unit and the audio capturing unit, in which the audio echo canceller unit includes a model of an acoustic wave. The model produces an echo estimate which is subtracted from a captured audio signal, which includes the echo. The audio presenting unit includes a sound producing device connected and controlled by a current controlled source.

Further, the communication system may include a compensator configured to provide an electrical damping for the sound producing device. The sound producing device is connected and controlled by a current controlled power source, and the model of the acoustic wave is a linear model.

Another object of the present invention is an audio communication system and method for transferring audio signals, including an audio presenting unit configured to produce a first sound wave; an audio capturing unit configured to capture a second sound wave; and an acoustic echo canceller unit connected to the audio presenting unit and the audio capturing unit, in which the audio echo canceller unit includes a model of an acoustic wave. The model produces an echo estimate which is subtracted from a captured audio signal, which includes the echo. The audio presenting unit includes a sound producing device connected and controlled by a weighted combination of voltage and current controlled sources.

Yet another object of the present invention is a video conference system including an audio presenting unit configured to produce a first sound wave; an audio capturing unit configured to capture a second sound wave; and an acoustic echo canceller unit connected to the audio presenting unit and the audio capturing unit, so that the audio echo canceller unit includes a model of an acoustic wave. The model produces an echo estimate which is subtracted from a captured audio signal which includes an echo. The audio presenting unit includes a sound producing device connected and controlled by a weighted combination of voltage and current controlled sources.

Another object of the present invention is a method for generating echo-free speech in a communication system, including: producing a sound wave by an audio presenting unit; capturing a sound wave by an audio capturing unit; canceling an echo by an acoustic echo canceller unit connected to the audio presenting unit and the audio capturing unit; producing an echo estimate by a model of an acoustic wave; subtracting the echo estimate from the captured sound wave which includes the echo; and controlling by a weighted combination of voltage and current control sources of sound producing device included into the audio presenting unit. In addition, the method of this object may control the sound producing device by the current controlled sources, when the model of the acoustic wave is a linear model.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a block diagram of a conventional conferencing system setup;

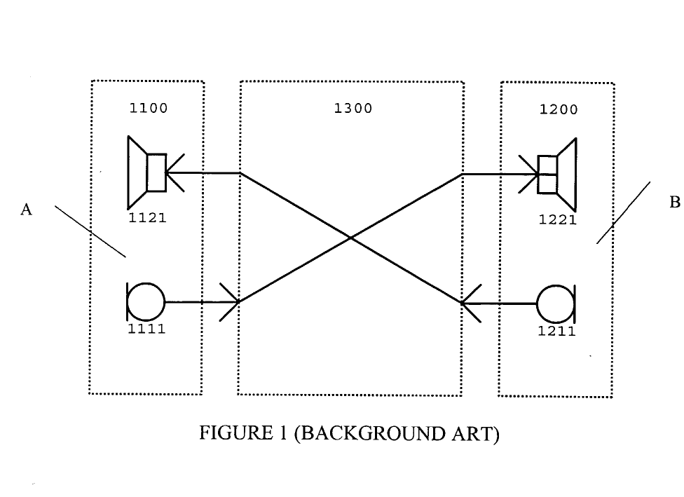

FIG. 2 is a block diagram of a video conferencing system setup according to the present invention;

FIG. 3 is a block diagram of an acoustic system and an acoustic echo canceller according to the present invention;

FIG. 4 is a block diagram of a digital processor, an amplifier, and a loudspeaker according to the present invention;

FIG. 5 is a block diagram of a power operational amplifier which controls a loudspeaker;

FIG. 6 is a block diagram of a loudspeaker controlled by a weighted combination of voltage and current controlled sources according to the present invention;

FIG. 7 is a block diagram of an alternate arrangement which controls the loudspeaker by a weighted combination of voltage and current controlled sources according to the present invention; and

FIG. 8 is a block diagram of a computer system that can be incorporated into the acoustic system according to the present invention.

### DESCRIPTION OF THE PREFERRED EMBODIMENT

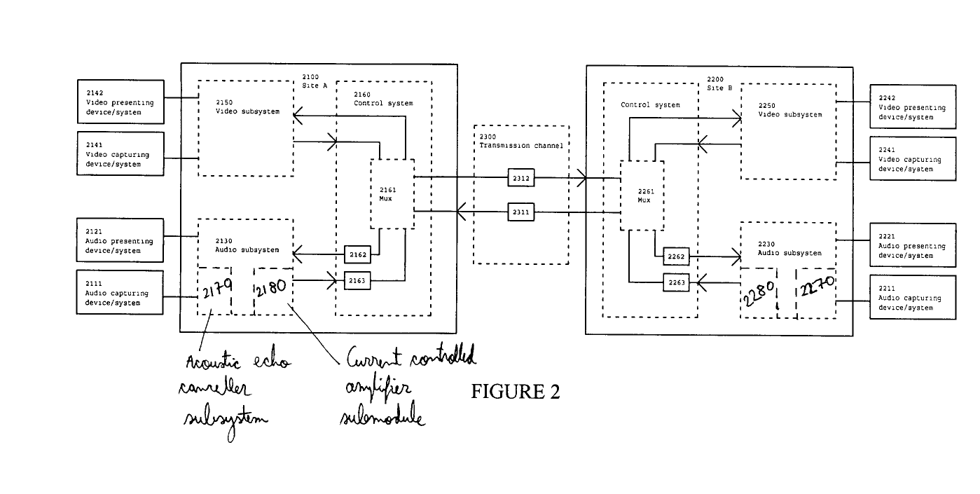

Referring now to the drawings, wherein like reference numerals designate identical or corresponding parts throughout the several views, FIG. 2 shows a video conferencing system. This system is distributed at two sites, A and B. The present invention can be applied to a setup distributed at more than two sites and also to a setup where only one participant has an acoustic system with a loudspeaker. As for the conferencing system setup, a video conferencing module can be distributed at more than two sites and also the system setup is functional when only one site has a loudspeaker. The video module has at site A a video capturing system 2141 that captures a video image and a video subsystem 2150 that encodes the video image. In parallel, a sound wave is captured by an audio capturing system 2111 and an audio subsystem 2130 encodes the sound wave to the acoustic signal. Due to processing delays in the video encoding system, the control system 2160 introduces additional delays to the audio signal by use of a lipsync delay 2163 so to achieve a synchronization between the video and audio signals. The video and audio signals are mixed together in a multiplexer 2161 and the resulting signal, the audio-video signal is sent over the transmission channel 2300 to site B.

The transmission channel 2300 and the audio subsystem 2230 at site B add further delays to the audio-video signal 2312. Another lipsync delay 2262 is added to site B in order to compensate for a video decoding delay that takes place in the video subsystem 2250 at site B. Subsequently, the audio and video signals, when presented to the audio and video presenting systems 2221 and 2242, respectively, are compensated for the delays described above. In addition to the video and audio signals captured at site A and presented at site B, the above description is valid for a video capturing device 2241 and an audio capturing device 2211 disposed at site B and for a video presenting device 2142 and an audio presenting device 2121 disposed at site A. In other words, the process described for capturing/reproducing the audio and video signals at one site and transmitting these signals to another site is also valid when the acoustic signals are transmitted from B to A.

Further, the audio signal presented by the audio presenting device 2221 is materialized as a sound wave at site B. Part of the sound wave presented at site B arrives to the audio capturing device 2211 either as a direct sound wave or as a reflected sound wave. Capturing the sound at site B and transmitting this sound back to site A together with the associated delays forms the echo. All delays described sums up to be considerable and therefore the quality requirements for an echo canceller in the video conferencing system are particularly high.

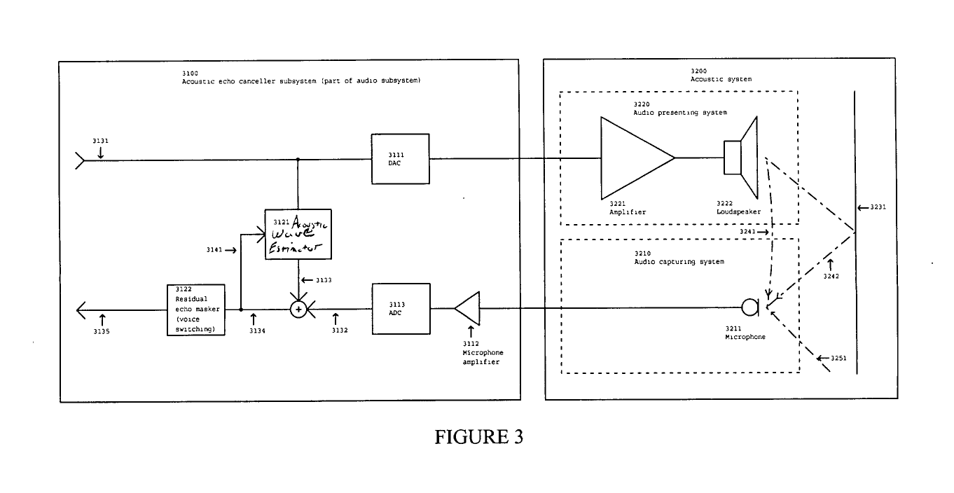

The audio subsystems 2130 and 2230 include an acoustic echo canceller subsystem 2170 and 2270, respectively, and a current control amplifier submodule 2180 and 2280, respectively. Each of the acoustic echo canceller subsystem 2170 and 2270 is connected to the audio capturing device 2111 and 2211, respectively. Each of the current control amplifiers submodule 2180 and 2280 is connected to the audio presenting device 2121 and 2221, respectively. A more detailed discussion of the acoustic echo canceller subsystem and the current control amplifier submodule is presented with reference to FIG. 3.

FIG. 3 shows the audio subsystem having an acoustic echo canceller subsystem 3100 and an acoustic system 3200. At least one of the participant sites has the acoustic echo canceller subsystem in order to reduce the echo in the communication system. The acoustic echo canceller subsystem 3100 is a full band model of a digital acoustic echo canceller but the present invention is applicable to all known echo cancellers, including subband echo cancellers (in which the audio signals are divided into several frequency bands, and where one or more of the processing blocks described are duplicated for each frequency band) and also analog echo cancellers. A full band model processes a complete audio band (e.g., up to 20 kHz; for video conferencing the band is typically up to 7 kHz, in audio conferencing the band is up to 3.4 kHz) of the audio signals directly. The audio signal 3131 coming from one site is converted by the acoustic echo canceller subsystem 3100 from the digital to the analog domain by the digital-to-analog converter 3111. The digitized acoustic signal enters the acoustic system 3200, particularly the amplifier 3221, and the loudspeaker 3222. The audio presenting system 3220, which includes the amplifier 3221 and the loudspeaker 3222, transforms the digitized audio signal to a sound wave and also introduces unwanted errors/nonlinearities.

The sound wave produced by the loudspeaker 3222, including the nonlinear components introduced by the acoustic system 3200, is captured by the microphone 3211, which is part of the audio capturing system 3210. The microphone 3211 captures the sound wave together with the nonlinear components either as the direct sound wave 3241 or as the reflected sound wave 3242. The reflected sound wave 3242 is produced by a reflection of the sound produced by the loudspeaker 3222 on a wall 3231. Other objects create full or partial reflection as well, such as movable objects like chairs or people. Although only one reflection is shown in FIG. 3, in reality many reflections are taking place depending on the environment where the sound is presented and also multi-reflections are usual, i.e., sound reflected by more than one object. In addition, any moving object reflects part of the sound produced by the loudspeaker 3222 and changes the reflection pattern around the microphone 3211.

Ideally, the microphone 3211 captures only the wanted sound wave 3251, normally produced by a person who speaks and uses the conferencing system to transmit a message from one site to another. As explained, the microphone 3211 captures not only the wanted sound wave 3251 but also the direct sound wave 3241 and the reflected sound wave 3242, and also a background noise produced around the microphone 3211. Once the microphone 3211 captures these sound waves, an acoustic signal is sent to a microphone amplifier 3112, and the acoustic signal is converted from the analog to digital domain by an analog-to-digital converter 3113. The digital signal 3132 represents the transformed value of the sound wave 3251, a linear transformation of the original acoustic signal 3131, and a nonlinear transformation of the original acoustic signal 3131. The linear transformation of the original acoustic signal 3131 was captured by the microphone 3211 as the direct sound wave 3241. The nonlinear component, which is substantial in conventional systems, is suppressed in the current invention at amplifier 3221.

A processor, or ASIC (Application Specific IC), embodies a model of an acoustic wave and this implements the acoustic wave estimator 3121 that is provided into the acoustic echo canceller subsystem 3100. This estimator 3121 receives the original acoustic signal 3131 and outputs an estimate (or negative or inverted estimate) acoustic echo 3133 of the original acoustic signal 3131. Thus, the acoustic signal 3134 is the acoustic signal produced by the microphone 3211 from which the estimated acoustic echo 3133 is subtracted, i.e., the wanted signal plus a residual echo obtained as a transformation of the original acoustic signal 3131. If the acoustic wave estimator 3121 embodies a linear model, the residual echo includes the complete nonlinear transfer function of the acoustic system 3200. A widely used model of the transformation performed by the acoustic system is implemented as a finite impulse response (FIR) filter. However, the present invention is applicable with other models as well.

The residual echo masker (also called nonlinear processor) 3122 receives the acoustic signal 3134 and removes most of the residual echo by introducing a time variant attenuation which may be frequency dependent. The time variant attenuation also attenuates the representation of the wanted signal 3251 and therefore the system is no longer completely duplex. The acoustic signal produced by the residual echo masker 3122 and transmitted to other site (or sites) 3135 is a tradeoff between no residual echo and transparent transmission of the wanted signal. Moreover, complete suppression of the wanted signal occurs if there is a need to minimize echo cancellations for original acoustic signal 3131, and vice versa.

In addition, whenever the acoustic environment changes, for example when a door opens, the acoustic wave estimator 3121 has to readapt itself to the new change. This is achieved with a feedback loop 3141 which monitors the errors and the changes in the environment. The loop 3141 feeds back a signal 3134 that represents the microphone signal from which the estimate acoustic echo is subtracted. One of the widely used algorithms for adapting the model to changed environments is the least means square algorithm (LMS). However, the present invention is applicable to other algorithms as well.

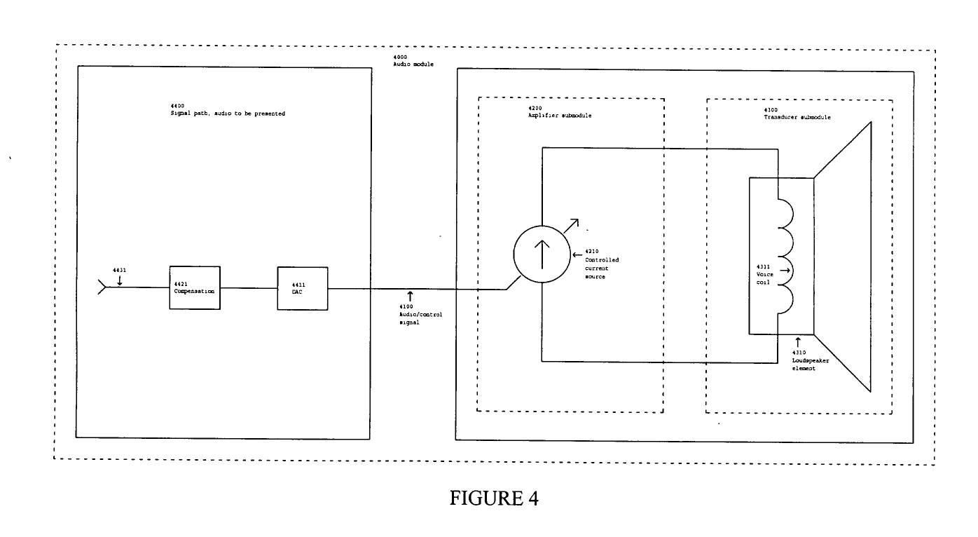

FIG. 4 presents in more detail the path followed by an audio signal 3131 (in FIG. 3) through the digital-to-analog converter 3111, the amplifier 3221, and the loudspeaker 3222. The audio presenting system 4000 includes a transducer submodule 4300, an amplifier submodule 4200, and a signal path of the audio signal to be presented 4400. The transducer submodule 4300 includes at least one loudspeaker element 4310 with a voice-coil element 4311 controlled by a current controlled source 4210 provided into the amplifier submodule 4200. The current controlled source 4210 works as a power amplifier for the loudspeaker element and the present invention is applicable for a setup with more than one current controlled source. The current controlled source can be implemented using many different approaches, for example using a power operational amplifier, or an audio amplifier with operational amplifier-like properties, as for example LM3886 from National Semiconductors. The signal path of the audio signal to be presented 4400 shows the audio signal 4431 (which is the same as the electrical audio signal 3131 in FIG. 3) being compensated by the compensator 4421 before being converted from the digital to analog domain by the module 4411, which is the same as module 3311 in FIG. 3.

By controlling the loudspeaker 4310 with the current controlled source 4210, the electrical damping is inherently lost. Without the electrical damping, any loudspeaker oscillates at its resonance frequency for some time after an audio signal is applied, reducing the audio quality/speech intelligibility. The electrical damping is achieved in the present invention by using a feedforward correction produced by the compensator 4421 and a digital compensation of the acoustic signal 4431. The compensator 4421 is implemented using a FIR or an infinite impulse response (IIR) filter, approximating the inverse of an impulse response of the combined loudspeaker and transducers 4200 and 4300. In the present system, the compensator 4421 may act also as a digital equalizer and for this function the compensator 4421 is implemented using a FIR filter.

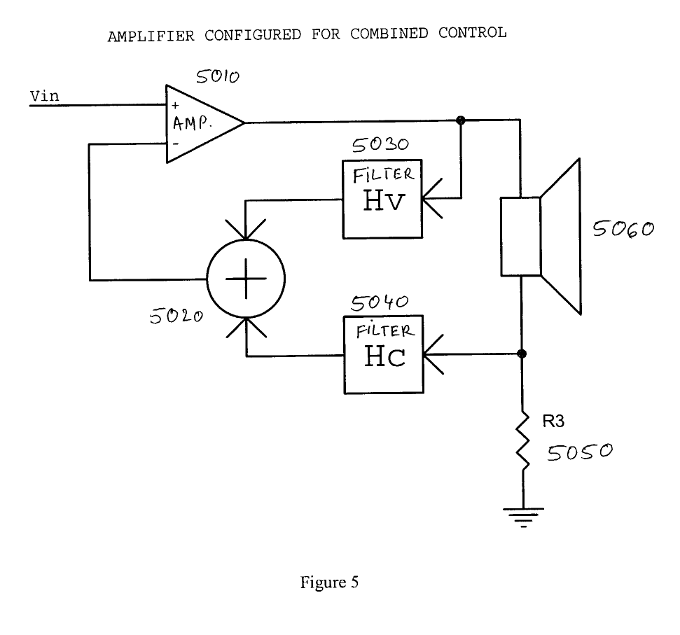

FIG. 5 is a block diagram of a power operational amplifier which controls the loudspeaker 5060. As discussed above, a problem with a current controlled source is that an impedance of the loudspeaker that is controlled by the respective source increases around the resonant frequency of the current controlled source. The increased impedance is not seen by a voltage controlled source, but the disadvantage of using the voltage controlled source is that high non-linearities are introduced by the loudspeaker, as discussed above. Therefore, the present inventor has recognized that by using a current controlled source with a voltage controlled source and by sending audio signals with a frequency close to the resonance frequency to the voltage controlled source and the other audio signal components to the current controlled source, the above-identified problem is solved.

The power amplifier 5010 is configured to act as a combined voltage and current controlled source. When acting as a combined voltage and current controlled source, input coming along two signal paths are summed together by adder 5020 to form a combined feedback loop. A first signal path transmits the output voltage of the amplifier to filter 5030 to be filtered and the output of the filter 5030 is input to adder 5020. An alternative to this approach is to measure a difference between terminals of the loudspeaker and feed the measured signals to filter 5030. A second signal path transmits a current measured after passing through the loudspeaker and filtered by the filter 5040 to the adder 5020. The measured current is proportional to the voltage across a current sensing device 5050. Selecting the filters 5030 and 5040 to act as low/high or pass/stop band filters, a weighted combination of input from the voltage and current controlled sources is achieved. Next, two examples of configuring the filters 5030 and 5040 are presented.

In a first example, the filter 5030 is configured as a lowpass filter and the filter 5040 is configured as a highpass filter so that both rolloff frequencies are selected to be above the resonance frequency of the loudspeaker. Thus, the acoustic system is voltage controlled for a frequency below the resonance frequency and current controlled for a frequency above the resonance frequency. Alternatively, in a second example, the filter 5030 is configured as a bandpass filter while the filter 5040 is configured as a bandstop filter. Thus, the passband and stopband are chosen to be around the resonance frequency of the loudspeaker and therefore the acoustic system is voltage controlled around the resonance frequency and current controlled for other frequencies. When the power amplifier described above and shown in FIG. 5 is used in a weighted control of voltage and current controlled sources to drive a load with a frequency dependent impedance (a loudspeaker) a ratio between the input from the current controlled source and the input from the voltage controlled source is influenced by the impedance curve of the loudspeaker. Compared to a nominal impedance, the impedance of the loudspeaker typically increases by 3 to 4 times around the resonance frequency and the current through the loudspeaker and the current sensing device 5050 is correspondingly lower compared to the voltage across the loudspeaker. For example, for a nominal impedance of 8 and a peak impedance of about 26: , at a maximum voltage output of 56 V, the current through the loudspeaker 5060 and the current sensing device 5050 is around 7 A for a frequency different the resonance frequency and around 2.15 A for a frequency around the resonance frequency. Thus, the input to the filter 5040 is low compared to the input to the filter 5030, and this difference in input determines if the system is voltage controlled around the resonance frequency. This difference in input is in addition to the transition determined by the properties of the filters 5030 and 5040 and reduces the need for higher order filters. When the input to filter 5040 is higher than the input to filter 5030, the acoustic system is current controlled and the frequency of the system is away from the resonance frequency.

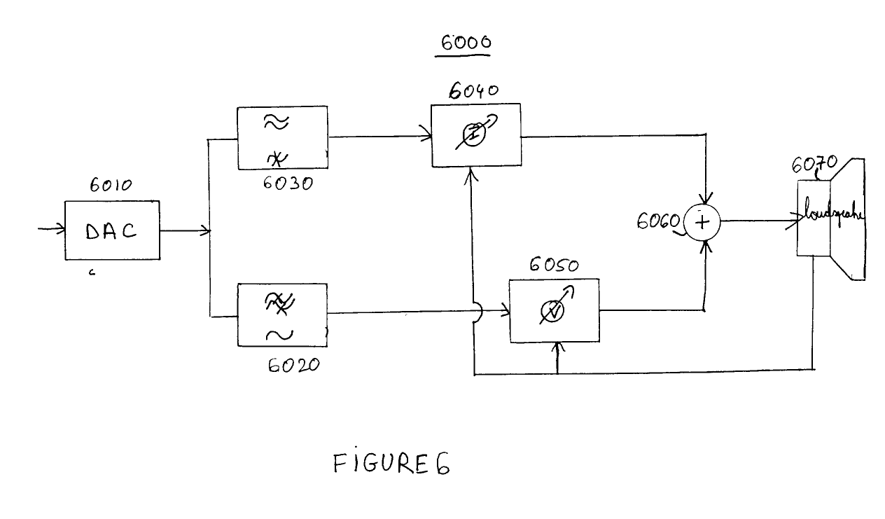

FIG. 6 is a block diagram of the loudspeaker 6070 controlled by a hybrid controller that provides a weighted combination of voltage and current controlled sources 6040 and 6050. The voltage controlled source 6040 is a power operational amplifier or alternatively an audio amplifier with operational amplifier-like properties, as for example LM3886 from National Semiconductors. The audio signal produced by the digital to analog converter 6010 is transmitted to a highpass filter 6030 which removes a low frequency part of the spectrum and allows only a high frequency part of the spectrum to go through. In the present embodiment, the highpass filter 6030 has a stopband below 80-100 Hz but it is noted the resonance frequency is loudspeaker dependent in addition to the dependency of the enclosure. The resonance frequency for woofers in the present invention is around 80-100 Hz, but the resonance frequency may vary considerably. A resonance frequency below 20 Hz is not uncommon in large loudspeakers/subwoofers, and resonance frequencies up to several kilohertz is common on discant elements/domes. The dome used in the present invention has a resonance frequency around 1600 Hz but the invention is not limited to this value. The large spectrum resonance frequencies does not affect the principle of the present invention as discussed above.

Alternatively, a bandpass filter may be used as a substitute for highpass filter 6030, where the stop band is 80 Hz to 100 Hz, or where a resonance condition is found to occur for the current source 6040 when used to drive the loudspeaker 6070. A lowpass (or bandpass) filter 6020 is provided for removing an upper part of the frequency spectrum and for allowing only a lower part (or limited range, e.g. 80-100 Hz) of the frequency spectrum. Alternatively, a digital signal may be employed to switch energy with a frequency higher than 80-100 Hz to the current controlled source 6040 and energy with a lower frequency range to the voltage controlled source 6050.

An adder 6060 combines the signals coming from the current controlled source 6040 and the voltage controlled source 6050 and sends the control signal to the loudspeaker 6070. In this way, the electrical damping necessary for the current controlled source when driving a loudspeaker is achieved by using the voltage controlled source when the frequency of the audio signal is close to the resonance frequency.

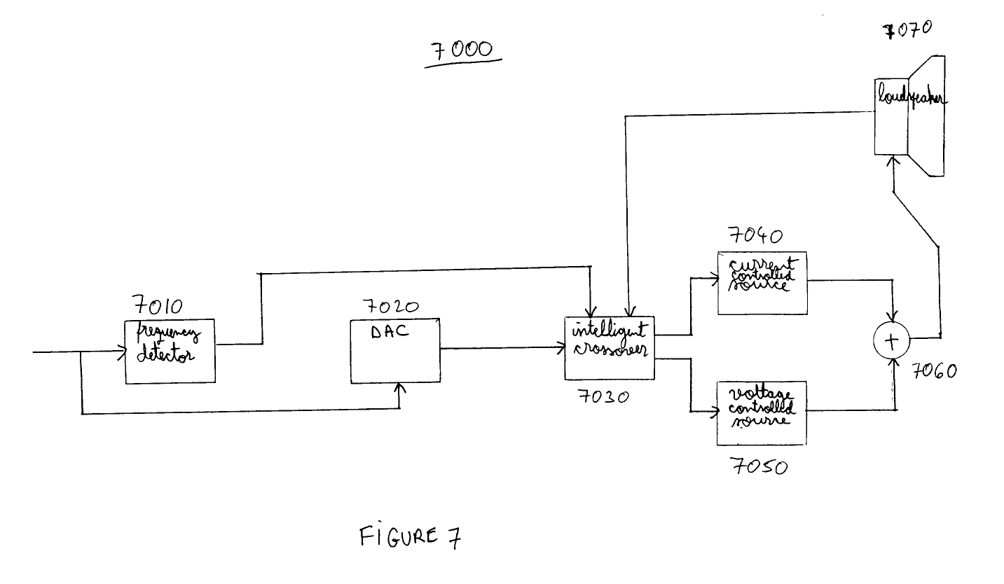

However, there are other possibilities to realize the electrical damping for the current controlled source. For example, FIG. 7 is a block diagram of a loudspeaker 7070 controlled by a current controlled source and a voltage controlled source when the signal inputted to the current and voltage controlled sources is produced by an intelligent crossover. In more detail, an audio signal is detected by a frequency detector 7010 and its frequency is evaluated and sent to the intelligent crossover 7030. The audio signal is converted by a digital to analog converter 7020 to a digital signal and then the digitized audio signal is transmitted to the intelligent crossover unit 7030. The intelligent crossover unit 7030, based on the frequency detected by the frequency detector 7010, the impedance sensed from the loudspeaker 7070 and the digitized audio signal produced by the digital to analog converter 7020 sends a control signal either to the current controlled source 7040 or to the voltage controlled source 7050, depending on the value of the frequency of the audio signal. If the frequency of the audio signal is around the resonance frequency of the current controlled source, in a ±10 Hz range, the intelligent crossover 7030 controls the voltage control source 7050 so that the electric damping is achieved. If the frequency of the audio signal is outside the resonance frequency of the current controlled source by more than a margin of ±10 Hz, then the intelligent crossover 7030 controls the current controlled source 7040. Depending on which source is controlled by the intelligent crossover 7030, the adder 7060 sends the respective control signal to the loudspeaker 7070.

The current controlled source advantageously reduces a harmonic distortion of the acoustical signal because the harmonic distortion in the loudspeaker element is caused by a nonlinearity of the B1 product, where B is the magnetic flux density in the loudspeaker magnet, and 1 is the length of the coil exposed to the magnetic flux density B. In the current controlled acoustic system, the harmonic distortion is reduced comparative with the voltage controlled acoustic system as the relation of the radiated sound pressure to the electrical input is proportional to B1 instead of (B1)². Another advantage of using the current controlled source for controlling the loudspeaker element is related to the sensitivity of the loudspeaker to the temperature of the voice-coil. The voice-coil has a positive temperature coefficient of resistance, which means that increasing the input power, the temperature of the voice-coil raises and accordingly the resistance of the voice-coil. With a constant voltage drive mechanism, as the temperature increases, the current decreases. The acoustic output is proportional to the current and therefore, the acoustic output decreases for the voice-coil controlled by a voltage source, because the audio power is time variant and the loudspeaker transfer function would be time variant as well. In the current controlled source setup, the current in the circuit is held constant, resulting in no change in the acoustic output and therefore the transfer function of the loudspeaker is time invariant.

Further, by using the compensator to introduce the electrical damping for the loudspeaker element, the oscillation of the loudspeaker at its resonance frequency is removed and the audio quality/speech intelligibility is improved. It is noted that the compensator 4421 is implemented in a digital signal processor, or digital circuit (as described with reference to FIGS. 5-7) and produces a digital compensation and not an electro-mechanical compensation.

The technical features of the present invention enhance the quality of the audio presenting system when used with an echo canceller because a performance of the echo canceller is improved due to the reduction of the harmonic distortion and the non-linearity of the acoustic system. Therefore, it is possible to use simple and inexpensive echo canceller systems that use linear models because the current controlled source controlling the loudspeaker produces a linear acoustic signal. It is also possible to control the loudspeaker by a weighted combination of voltage and current controlled sources as explained with reference to FIGS. 5-7. Consequently, standard, low cost loudspeakers are used with the same results as the highly sophisticated and expensive loudspeakers and in addition, the need for voice switching is decreased. It is known that the voice switching affects the sound spectrum as a whole and the reduction of voice switching leads to better sound quality.

Even more, the conferencing system of the present invention does not need to readapt to a changed sensitivity of the loudspeaker caused by a varying signal power applied to the loudspeaker. In the voltage controlled system, the echo compensator has to track and adapt to the time variance of the sensitivity. The echo canceller will adapt whenever a signal is detected by the loudspeaker but still fails to completely cancel a residual echo as explained next.

With reference to FIG. 2, if a person talks at site A for a period of time, the temperature of the voice-coil of the loudspeaker at site B increases. The echo canceller adapts to the sensitivity of the loudspeaker with an increased temperature of the voice-coil, and during the time the person talks continuously at site A, little residual is present. If the person at site A stops talking, the temperature of the voice-coil decreases and the sensitivity of the loudspeaker changes. When the person talks again, the echo canceller uses the model produced before the person ceased to talk, i.e., uses the model of the high temperature voice-coil. However, the voice-coil is now at a lower temperature, and the sensitivity of the loudspeaker has increased. Therefore, the echo canceller underestimates the echo, producing the residual echo.

A similar but opposite problem arises if a person talks at a reduced volume level first. The echo canceller adapts to a moderate high temperature voice-coil. When the person starts talking louder, the voice-coil will constantly heat up (until it will finally stabilize at a high temperature), and for the time the coil gets warmer, the sensitivity decreases. Thus, the echo canceller overestimates the echo, resulting in the production of residual echo.

In addition, the learning curve of the echo compensator is considerably longer than the time constant of the fluctuations due to adaptation and before the echo compensator readjusts itself, there will be a gain mismatch. Further, a moderate increase of the temperature in the voice-coil of the loudspeaker of 100° C. reduces the output/gain by 3 dB. A gain mismatch of only 3 dB reduces the possible echo canceling depth to only 10.7 dB or 7.7 dB, depending on whether the coil has heated up or cooled down, respectively.

Faster adaptation to changed acoustic environment for the acoustic system as the nonlinear components of the residual echo are reduced is highly desirable and achievable by the present invention by using a weighted combination of input from the voltage and current controlled sources. Any moving object in a room where the acoustic system resides changes the reflection pattern for the propagation of a sound wave, and thereby the acoustic environment. Typical examples are a moving person or a door which opens or closes. The nonlinear components of the acoustic signal 3131, which are incorporated in the signal 3134, negatively influence the feedback loop 3141. The nonlinear components have especially a negative impact as they are correlated with the linear echo. This is due to the harmonic properties of the audio part (speech/music), normally presented by the audio presenting device. Therefore, as discussed, the present invention is capable of faster adapting to changed acoustic environments and improved sound quality, by reducing the harmonic distortion and the nonlinear components of the audio signal.

The foregoing discussion discloses and describes merely an exemplary embodiment of the present invention. As will be understood by those skilled in the art, the present invention may be embodied in other specific forms without departing from the spirit or essential characteristics thereof. Accordingly, the disclosure of the present invention is intended to be illustrative, but not limiting, of the scope of the invention, which is set forth in the following claims. The entire contents of U.S. Pat. Nos. 5,937,060, 5,737,408 and 6,198,819 are incorporated herein by reference. Also, the entire contents of the articles “Non-Linear Acoustic Echo Cancellation with Fast Converging Memoryless Preprocessor” by Stenger and Kellerman and “Electronic Damping for Dynamic Drivers in Vented Enclosures,” J. Audio Eng. Soc., Vol. 47, No. 1/2, January/February, by Hsu and Poornima are incorporated herein by reference.

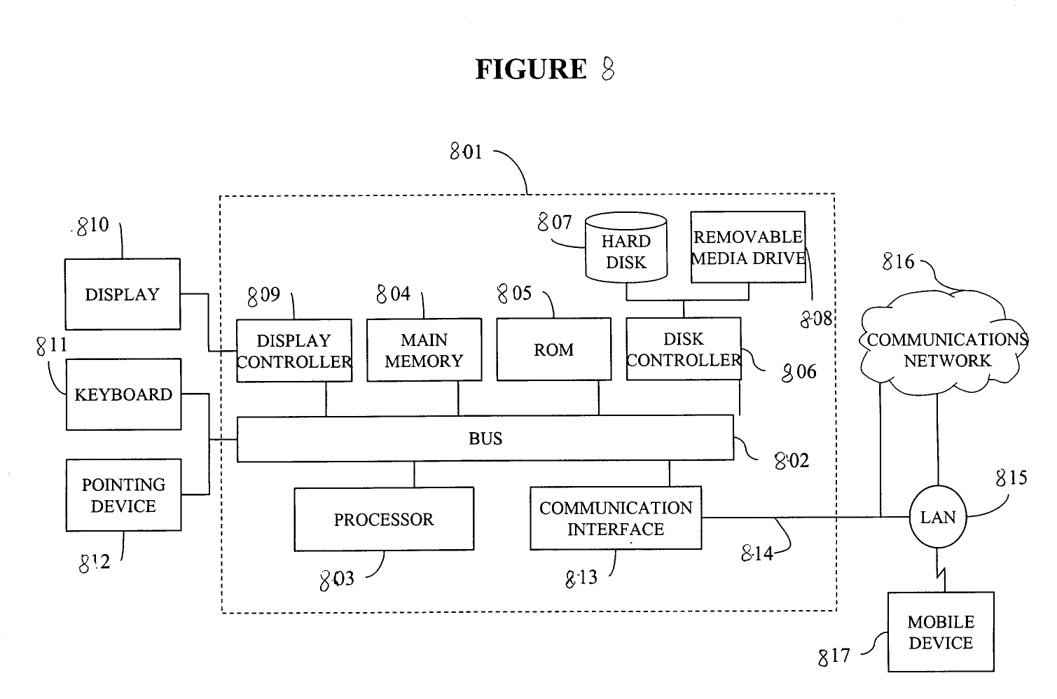

FIG. 8 illustrates a computer system 801 upon which an embodiment of the present invention may be implemented to handle the control operations discussed above. In more detail, the audio echo canceller unit may include parts or all the computer system 801 for implementing the model of the acoustic wave, for producing the echo estimate, or for various mathematical manipulations applied to the sound wave. The computer system 801 includes a bus 802 or other communication mechanism for communicating information, and a processor 803 coupled with the bus 802 for processing the information. The computer system 801 also includes a main memory 804, such as a random access memory (RAM) or other dynamic storage device (e.g., dynamic RAM (DRAM), static RAM (SRAM), and synchronous DRAM (SDRAM)), coupled to the bus 802 for storing information and instructions to be executed by processor 803. In addition, the main memory 804 may be used for storing temporary variables or other intermediate information during the execution of instructions by the processor 803. The computer system 801 further includes a read only memory (ROM) 805 or other static storage device (e.g., programmable ROM (PROM), erasable PROM (EPROM), and electrically erasable PROM (EEPROM)) coupled to the bus 802 for storing static information and instructions for the processor 803.

The computer system 801 also includes a disk controller 806 coupled to the bus 802 to control one or more storage devices for storing information and instructions, such as a magnetic hard disk 807, and a removable media drive 808 (e.g., floppy disk drive, read-only compact disc drive, read/write compact disc drive, compact disc jukebox, tape drive, and removable magneto-optical drive). The storage devices may be added to the computer system 801 using an appropriate device interface (e.g., small computer system interface (SCSI), integrated device electronics (IDE), enhanced-IDE (E-IDE), direct memory access (DMA), or ultra-DMA).

The computer system 801 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 801 may also include a display controller 809 coupled to the bus 802 to control a display 810, such as a cathode ray tube (CRT), for displaying information to a computer user. The computer system includes input devices, such as a keyboard 811 and a pointing device 812, for interacting with a computer user and providing information to the processor 803. The pointing device 812, for example, may be a mouse, a trackball, or a pointing stick for communicating direction information and command selections to the processor 803 and for controlling cursor movement on the display 810. In addition, a printer may provide printed listings of data stored and/or generated by the computer system 801.

The computer system 801 performs a portion or all of the processing steps of the invention in response to the processor 803 executing one or more sequences of one or more instructions contained in a memory, such as the main memory 804. Such instructions may be read into the main memory 804 from another computer readable medium, such as a hard disk 807 or a removable media drive 808. One or more processors in a multi-processing arrangement may also be employed to execute the sequences of instructions contained in main memory 804. In alternative embodiments, hard-wired circuitry may be used in place of or in combination with software instructions. Thus, embodiments are not limited to any specific combination of hardware circuitry and software.

As stated above, the computer system 801 includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system 801, for driving a device or devices for implementing the invention, and for enabling the computer system 801 to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code devices of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

The term “computer readable medium” as used herein refers to any medium that participates in providing instructions to the processor 803 for execution. A computer readable medium may take many forms, including but not limited to, non-volatile media, volatile media, and transmission media. Non-volatile media includes, for example, optical, magnetic disks, and magneto-optical disks, such as the hard disk 807 or the removable media drive 808. Volatile media includes dynamic memory, such as the main memory 804. Transmission media includes coaxial cables, copper wire and fiber optics, including the wires that make up the bus 802. Transmission media also may also take the form of acoustic or light waves, such as those generated during radio wave and to infrared data communications.

Various forms of computer readable media may be involved in carrying out one or more sequences of one or more instructions to processor 803 for execution. For example, the instructions may initially be carried on a magnetic disk of a remote computer. The remote computer can load the instructions for implementing all or a portion of the present invention remotely into a dynamic memory and send the instructions over a telephone line using a modem. A modem local to the computer system 801 may receive the data on the telephone line and use an infrared transmitter to convert the data to an infrared signal. An infrared detector coupled to the bus 802 can receive the data carried in the infrared signal and place the data on the bus 802. The bus 802 carries the data to the main memory 804, from which the processor 803 retrieves and executes the instructions. The instructions received by the main memory 804 may optionally be stored on storage device 807 or 808 either before or after execution by processor 803.

The computer system 801 also includes a communication interface 813 coupled to the bus 802. The communication interface 813 provides a two-way data communication coupling to a network link 814 that is connected to, for example, a local area network (LAN) 815, or to another communications network 816 such as the Internet. For example, the communication interface 813 may be a network interface card to attach to any packet switched LAN. As another example, the communication interface 813 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 813 sends and receives electrical, electromagnetic or optical signals that carry digital data streams representing various types of information.

The network link 814 typically provides data communication through one or more networks to other data devices. For example, the network link 814 may provide a connection to another computer through a local network 815 (e.g., a LAN) or through equipment operated by a service provider, which provides communication services through a communications network 816. The local network 814 and the communications network 816 use, for example, electrical, electromagnetic, or optical signals that carry digital data streams, and the associated physical layer (e.g., CAT 5 cable, coaxial cable, optical fiber, etc). The signals through the various networks and the signals on the network link 814 and through the communication interface 813, which carry the digital data to and from the computer system 801 maybe implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 801 can transmit and receive data, including program code, to through the network(s) 815 and 816, the network link 814 and the communication interface 813. Moreover, the network link 814 may provide a connection through a LAN 815 to a mobile device 817 such as a personal digital assistant (PDA) laptop computer, or cellular telephone.

Obviously, numerous modifications and variations of the present invention are possible in light of the above teachings. It is therefore to be understood that within the scope of the appended claims, the invention may be practiced otherwise than as specifically described herein.

What we claim is:

1. An audio communication system for transferring audio signals, comprising:
an audio presenting unit configured to produce a sound wave;
an audio capturing unit configured to capture another sound wave; and
an acoustic echo canceller unit connected to said audio presenting unit and said audio capturing unit,
wherein the audio echo canceller unit includes a circuit configured to implement a model of an acoustic wave, said circuit configured to produce an echo estimate and subtract the echo estimate from the sound wave captured by said audio capturing unit, said sound wave including an echo, and
wherein the audio presenting unit includes a sound producing device connected and controlled by a current controlled source.

2. An audio communication system according to claim 1, further comprising a compensator configured to provide electrical damping for the sound producing device.

3. An audio communication system according to claim 1, further comprising a voltage controlled source, wherein the sound producing device is driven by a weighted combination of input from the voltage controlled source and the current controlled-source.

4. An audio communication system according to claim 1, wherein the model of the acoustic wave is a linear model.

5. An audio communication system according to claim 1, wherein the circuit includes a processor and the current controlled source is a power operational amplifier.

6. An audio communication system according to claim 1, wherein the sound producing device is a loudspeaker.

7. An audio communication system according to claim 6, wherein the audio presenting unit includes an amplifier connected by an interface to the loudspeaker.

8. An audio communication system according to claim 3, wherein said interface is adapted so that the loudspeaker is controlled by the weighted combination of voltage and current controlled sources.

9. An audio communication system according to claim 1, further comprising a video conferencing module, wherein said audio communication system is configured as an audio portion of a video conferencing system.

10. An audio presenting system which is part of a video conferencing system, comprising:
an audio presenting unit configured to produce a sound wave;
an audio capturing unit configured to capture another sound wave; and
an acoustic echo canceller unit connected to said audio presenting unit and said audio capturing unit,
wherein the audio echo canceller unit includes a circuit configured to implement a model of an acoustic wave, said circuit configured to produce an echo estimate and subtract the echo estimate from the sound wave captured by said audio capturing unit, said sound wave including an echo, and
wherein the audio presenting unit includes a sound producing device connected and controlled by a current controlled source.

11. An audio presenting system according to claim 10, further comprising a compensator configured to provide electrical damping for the sound producing device.

12. An audio presenting system according to claim 10, further comprising a voltage controlled source, wherein the sound producing device is driven by a weighted combination of input from the voltage controlled source and the current controlled source.

13. An audio presenting system according to claim 10, wherein the sound producing device is a loudspeaker and the current controlled source is a power operational amplifier.

14. An audio presenting system according to claim 13, wherein the audio presenting unit includes an amplifier connected by an interface to the loudspeaker.

15. An audio presenting system according to claim 14, wherein said interface is adapted so that the loudspeaker is controlled by the weighted combination of voltage and current controlled sources.

16. A method for generating echo-free audio in an audio communication system, comprising steps of:
producing a sound wave by an audio presenting unit;
capturing another sound wave by an audio capturing unit;
canceling an echo by an acoustic echo canceller unit connected to said audio presenting unit and said audio capturing unit;
producing an echo estimate by a circuit configured to implement a model of an acoustic wave;
subtracting the echo estimate from the sound wave captured by said audio capturing unit, said sound wave including an echo; and
controlling by a current controlled source a sound producing device included into the audio presenting unit.

17. A method according to claim 16, wherein the controlling is effected by a weighted combination of input from the voltage controlled source and the current controlled source.

18. A method according to claim 16, wherein producing a sound wave further includes providing electrical damping for the sound producing device.

19. A method according to claim 16, wherein the model of the acoustic wave is a linear model.

20. A method according to claim 16, wherein the audio communication system is an audio portion of a video conference system.

21. An audio presenting device for updating an installed communication system, comprising:
an audio presenting unit configured to produce a sound wave;
an audio capturing unit configured to capture another sound wave; and
an acoustic echo canceller unit connected to said audio presenting unit and said audio capturing unit,
wherein the audio echo canceller unit includes a circuit configured to implement a model of an acoustic wave, said circuit configured to produce an echo estimate and subtract the echo estimate from the sound wave captured by said audio capturing unit, said sound wave including an echo, and
wherein the audio presenting unit includes a sound producing device connected and controlled by a current controlled source.

22. An audio presenting device according to claim 21, further comprising a compensator configured to provide electrical damping for the sound producing device.

23. An audio presenting device according to claim 21, further comprising a voltage controlled source, wherein the sound producing device is driven by a weighed combination of input from the voltage controlled source and the current controlled source.

24. An audio presenting device according to claim 21, wherein the model of the acoustic wave is a linear model.

25. An audio presenting device according to claim 21, wherein the circuit includes a processor and the current controlled source is a power operational amplifier.

26. An audio presenting device according to claim 21, wherein the sound producing device is a loudspeaker.

27. An audio presenting device according to claim 21, wherein the audio presenting unit includes an amplifier connected by an interface to the loudspeaker.

28. An audio presenting device according to claim 27, wherein said interface is adapted so that the loudspeaker is controlled by the weighted combination of voltage and current controlled sources.

29. An audio presenting device according to claim 21, wherein said installed communication system is a video communication system.

30. A method of upgrading installed communication system, comprising steps of:
producing a sound wave by an audio presenting unit;
capturing another sound wave by an audio capturing unit;
canceling an echo by an acoustic echo canceller unit connected to said audio presenting unit and said audio capturing unit;
producing an echo estimate by a circuit configured to implement a model of an acoustic wave;
subtracting the echo estimate from the sound wave captured by said audio capturing unit, said sound wave including an echo; and
controlling by a current controlled source said sound producing device included in the audio presenting unit.

31. A method according to claim 30, wherein the controlling is effected by a weighted combination of input from the voltage controlled source and the current controlled source.

32. A method according to claim 30, wherein said installed communication system is a video communication system.
